(19) United States
(12) Patent Application Publication
(10) Pub. No.: US 2025/0082539 A1
(43) Pub. Date: Mar. 13, 2025

(54) INTRAGASTRIC MAGNETIC DEVICE AND EXTERNAL STIMULATOR

(71) Applicant: Appetec, Inc., Macon, GA (US)

(72) Inventors: Shahriar Sedghi, Macon, GA (US); Sheng-Chiang Lee, Macon, GA (US)

(73) Assignee: Appetec, Inc., Macon, GA (US)

(21) Appl. No.: 18/741,500

(22) Filed: Jun. 12, 2024

Related U.S. Application Data
(63) Continuation-in-part of application No. 17/491,450, filed on Sep. 30, 2021, now Pat. No. 12,016,791.

(51) Int. Cl. A61H 23/00

(52) U.S. Cl. CPC A61H 23/00; A61H 2201/0103; A61H 2201/5005

(57) ABSTRACT

The present invention further discloses intragastric medical devices which may be implanted within a patient's body without surgery. The intragastric device can be controlled remotely with external devices using the forces of magnetic attraction and repulsion. The present disclosure is further directed towards a vibrating implant and an associated external vibrating magnet. The implant device is designed to offer a novel approach to vibration therapy and may be used to induce a feeling of satiety in a patient.

### CROSS-REFERENCE TO RELATED APPLICATION

This patent application claims priority to U.S. patent application Ser. No. 17/491,450, filed on Sep. 30, 2021, and entitled “Intragastric Magnetic Device and Delivery System.” The disclosure of the prior application is considered part of and is incorporated by reference into this patent application.

### BACKGROUND

Obesity is a major medical problem affecting millions of people. It is generally considered that obesity is a food addiction problem.

Obese patients currently undergo several types of invasive surgery to either staple or tie off portions of the stomach, small intestine, and/or bypass portions of the same. The goal is to reduce the amount of food desired by the patient. Current methods for achieving these results include laparoscopic banding, surgical bypass, and gastric stapling. These methods often necessitate incisions and general anesthesia and may cause long- or short term complications.

Less invasive endoscopic procedures are also used to assist weight loss and have primarily focused on placement of a balloon or other space-occupying device in the patient's stomach to provide a continual feeling of fullness and consequential reduction in food intake, often in conjunction with behavioral modification programs.

To accomplish these procedures, an endoscope is generally utilized to guide the space-occupying device through the patient's mouth, down the esophagus, and into the stomach before relinquishing control of the device for some 4-12 months, and endoscopically retrieving it thereafter. Additionally, air leakiness especially is a major problem with current balloons. It is typically the case that the smaller the balloon, the higher the pressure with more leakiness.

There exists a need in the art to address the problems described above.

### SUMMARY

The present disclosure provides an intragastric implant capable of inducing the feeling of satiety from inside the stomach, together with a delivery device for delivering said implant.

In addition to patients who may otherwise be treated surgically as morbidly obese, the present disclosure provides systems and methods of providing minimally invasive weight loss procedures for patients who are only moderately overweight or obese, reducing the risks associated with more invasive procedures.

The present invention further discloses intragastric medical devices which may be implanted within a patient's body without surgery. The intragastric device can be controlled remotely with external devices using the forces of magnetic attraction and repulsion. The present disclosure is further directed towards a vibrating implant and an associated external vibrating magnet. The implant device is designed to offer a novel approach to vibration therapy and may be used to induce a feeling of satiety in a patient.

Although specific advantages have been enumerated above, various embodiments may include some, none, or all of the enumerated advantages. Additionally, other technical advantages may become readily apparent to one of ordinary skill in the art after review of the following figures and description.

### BRIEF DESCRIPTION OF THE DRAWINGS

FIG. 1 is a perspective view of the inner core of a magnetic intragastric device.

FIG. 2A is a cross-sectional view of a magnetic intragastric device.

FIG. 2B is a perspective view of a magnetic intragastric device.

FIG. 3A and FIG. 3B are perspective views of a delivery device for the intragastric device.

FIGS. 4A and 4B show views of a poron/plastic covered magnet for impact absorption.

FIGS. 5A and 5B show views a magnet within a plunger system.

FIG. 6 shows a view of storage box with sensor/alarm for remote monitoring

FIG. 7 is a cross sectional view of an implant device according to an embodiment of the present disclosure.

FIG. 8 is an external vibrating magnet according to an embodiment of the present disclosure.

FIG. 9 of the present disclosure relates to an innovative massage vibrator 900.

FIG. 10 discloses an external device according to an embodiment of the present disclosure.

FIG. 11 illustrates EMS electrodes for stimulation, along with EMS control electronics integrated into a belt.

FIG. 12 illustrates an air bladder integrated into a belt, along with an air control pump and vent.

FIG. 13 illustrates a vibrator embodiment where a disk-shaped mechanical vibrator is housed in the belt.

### DETAILED DESCRIPTION

It should be understood at the outset that, although exemplary embodiments are illustrated in the figures and described below, the principles of the present disclosure may be implemented using other techniques. The present disclosure should in no way be explicitly limited to the exemplary implementations and techniques illustrated in the drawings and described below. Additionally, unless otherwise specifically noted, articles depicted in the drawings are not necessarily drawn to scale.

With specific reference to the embodiments and figures in detail, it is stressed that the particulars presented are by way of example for purposes of illustrative discussion of embodiments of the present invention only and are presented to provide what is believed to be the most readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

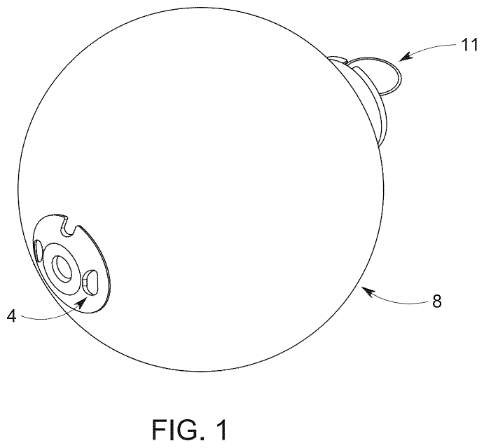

An intragastric device is presented in FIG. 1, which shows an outer view of a tubular intragastric device in its default deployed state. The intragastric device has a balloon 8 with a suture wire 11 appearing on a distal side of the balloon and a valve cover 4 appearing on the distal side.

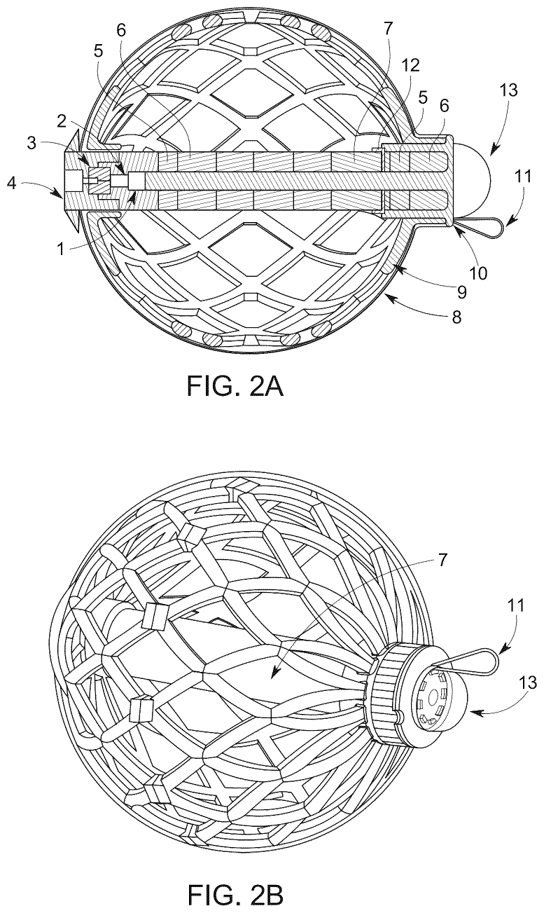

The intragastric device 100 is also presented in FIG. 2A, which shows a cross sectional view of a tubular intragastric device in its default deployed state. A pair of magnets 8 and 9 are located on the distal side, within a magnet enclosure cap 12. A core inner tube 1 goes through the middle of the device and attaches to the valve housing 2, with a septum 3 appearing at the end of the valve housing. Valve cover 4 is connected to the valve housing 2. A single piece stent 9 is placed within the balloon. An outer core tube 7 appears outside the inner core tube 1. Also provided is appendage 13. FIG. 2 B shows a perspective view of an intragastric device.

According to an embodiment, appendage 13 further comprises a sensor. The optional sensor can monitor drug delivery, according to this embodiment. In alternative embodiments, the sensor could monitor acidity, motility, disease, and other physiologic functions such as a pressure sensor. Thus, it can be appreciated that the function and usage of the balloon can be monitored from outside. By having pressure sensors, the patient or an AI system could identify which area of the stomach at which pressure. gives the best desired outcome.

In a further embodiment, the sensor could monitor movement of the device According to an embodiment, the sensor comprises an accelerometer. According to another embodiment, the sensor further comprises a hardware-based gyroscope. The sensor could further comprise a camera. The camera can be used long term to monitor healing of an ulcer or response to treatment of cancer or for chronic GI bleed, as examples. The sensor is operable to monitor linear acceleration, significant motion, rotation vector, step counter, and step detector sensors, via methods that are either hardware-based or software-based, as understood with such sensors currently available.

The sensor can be configured to measure and store data including optical data and be retrieved when the balloon is moved endoscopically and/or use a remote monitor to deliver data.

In an alternative embodiment, appendage 13 further comprises a drug delivery system, along with a remote monitoring system which is responsible for administering a drug contained in the appendage 13.

According to an embodiment, the stent 9 comprises a mesh-like pattern.

It can be appreciated that in a collapsed state, the stent can be placed within the delivery device.

In an alternative embodiment, a single magnet may be used instead of multiple magnets. The magnets comprise neodymium magnetics according to an embodiment.

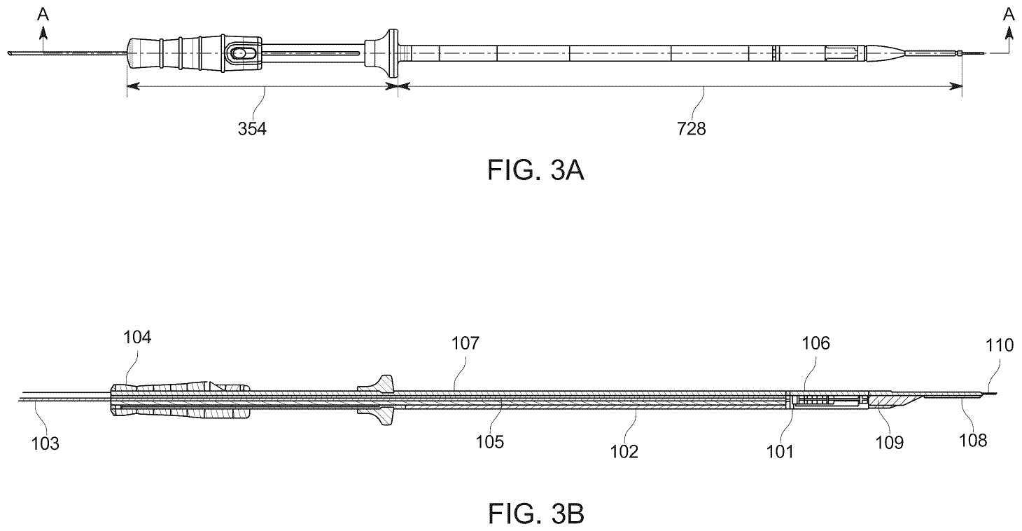

A first embodiment of a delivery device is presented in FIGS. 3A and 3B. An instagastric device in collapsed state is placed within a device deployment holder. A guide wire 110 runs though the dilator tip and is intended to guide the intagastric device 101 through an esophagus. The guide wire is surrounded by a guide wire tube 119. Also included is an inflation tube assembly 103, a sheath 102, a dilator tip 108, and a silicone support tube 105.

The collapsed state disclosed in FIGS. 3A and 3B is used during delivery of the intragastric device into the stomach of the patient. The spring loaded stent expands the intagastric device upon insertion into a patient's stomach, such that the stent is expanded into a balloon like structure. That is, once the device exits the esophagus and enters the stomach, the device's resilient and flexible structure reverts to its natural expanded state, in this case a balloon shape.

In one embodiment, the system is sealed so upon deployment it can't self-expand. Instead, an inflation tube is provided to expand the system. Once the inflation catheter tip is opened, the implant can self-expand or be aided in expansion by injection of air.

In a further novel embodiment, there is a small magnet on the other side of the tube that is attracted to the main set of magnets. It can be appreciated that this helps the frame to expand and acquire its structure. This feature is important as the delivery device is held inside a delivery tube for a period of time, thus it many take a while for the frame to fully acquire an expanded shape without this extra small magnet.

In a further embodiment, a silicone membrane is outside the frame and loose. Hence the device is a zero/low pressure system that does not leak air through silicone or through the valve. The primary function of the silicone membrane outside of the frame is to prevent food getting stuck in the frame.

In a further embodiment, an external device containing one or more magnets are provided to operate the system, together with safety features.

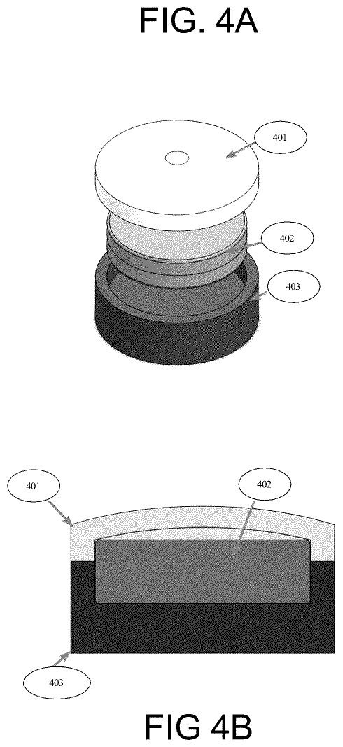

FIGS. 4A and 4B show a poron/plastic covered magnet for impact absorption. A magnet 402 is placed within a poron foam container 403 with a plastic upper case 401.

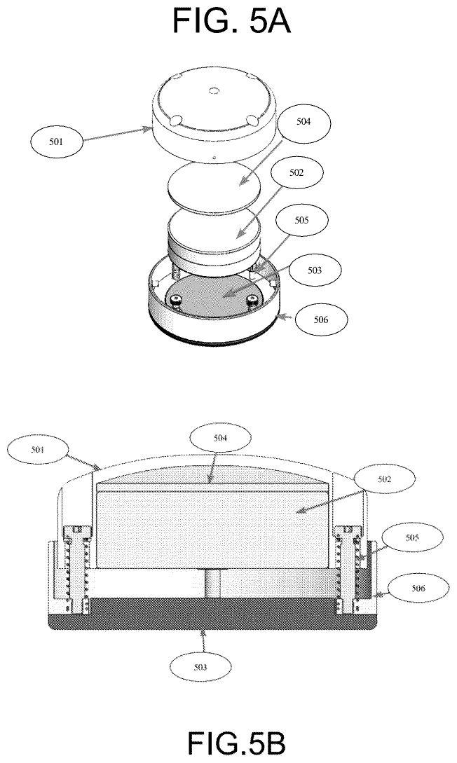

FIGS. 5A and 5B show a magnet 502 within a plunger system that has to be pushed down to be “activated”. When released by hand, the springs 505 push it back up creating space and sharply dropping magnetic force. The magnet 502 is placed within a poron foam container 503 with a plastic upper case 501.

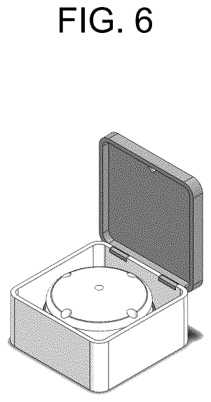

FIG. 6 shows a storage box with sensor/alarm for remote monitoring and to alert the user that the external magnet is not properly stored. In a further embodiment, RF sensors are added on the external magnet too to show how often they are being used.

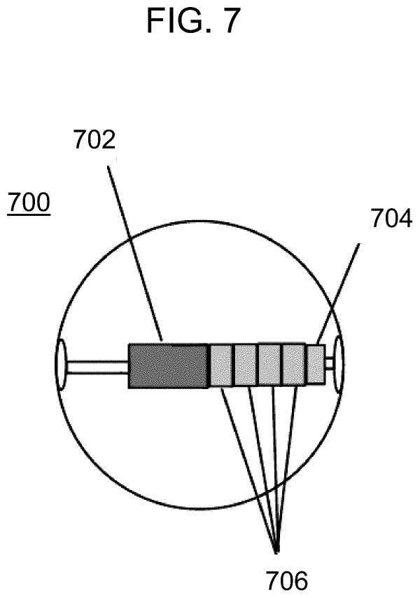

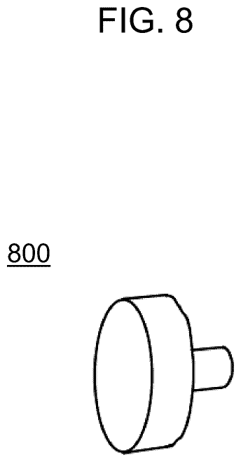

FIG. 7 is a cross sectional view of an implant device 700 according to an embodiment of the present disclosure. According to an embodiment of the present disclosure, the implant device 700 comprises several key components. These include an axial vibrator 702 that vibrates in the axial direction, and a set of electronics 704 that include a magnetically activated switch and a battery. The electronics 704 are positioned strategically between the internal magnet and the external magnet device (see FIG. 8 for an example of an external magnet device), facilitating magnetic activation.

Further embodiments of the implant device focus on ensuring compactness and ease of use. The design allows for easy delivery and removal of the implant, which is crucial for practical applications. Although the control circuit can be miniaturized to a great extent, the battery and motor will inevitably occupy more space. Therefore, the design balances between miniaturization and the functional requirements of housing the battery and motor, by placing the electrical control circuit directly next to the magnets.

In another embodiment, the implant can be customized for various applications such as in coordination with an app. The versatility of the design allows for modifications to suit specific needs, such as different vibration frequencies or intensities. The device's magnetic activation feature also enables it to be operated without the need for invasive procedures to turn it on or off.

The implant device can also be integrated with smart technologies for enhanced functionality. For instance, it could include sensors to monitor the implant's position and operation, providing real-time feedback to the user or healthcare provider. Additionally, the device could be connected to a smartphone app to control and customize vibration settings, ensuring optimal performance tailored to individual needs.

The activation mechanism for the device can be either mechanical or electrical. In the mechanical approach, according to an embodiment of the present disclosure, activation occurs through the compression force between the internal and external magnets. In the electrical approach, the system uses a magneto resistive component whose electric resistance varies with magnetic fields. According to an embodiment, the switch can be turned on when the magnetoresistance falls within the range corresponding to the “captured” scenario between the implant and the external magnet.

An additional embodiment involves the external device, which features a robust and safe design, to interact with the implant and activate it, either by a patient or by a medical professional. According to an embodiment, an external magnet is encased in a reinforced plastic case that is attached to the shaft of a massage vibrator. For added safety, the plastic case is wrapped with Poron or an equivalent material on the side and the face. This construction ensures both durability and user safety during operation. The external device works in tandem with the implant to provide the necessary magnetic activation and subsequent vibration.

In one specific embodiment, the implant includes a switch that activates vibration upon magnetic capture. This activation can be triggered by either the attraction or squeezing force between the internal and external magnets, or by the change in the magnetic field using magneto resistive materials. These materials exhibit a change in electrical resistance when exposed to a magnetic field. This second method may be preferred in scenarios where it is desirable to move the implant while it is vibrating. The external magnet, when brought close enough, induces sufficient change in magnetoresistance for the control circuit to recognize and activate the device.

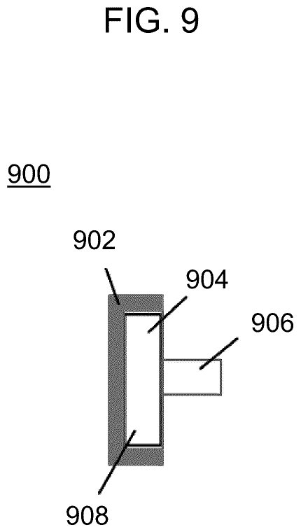

FIG. 9 of the present disclosure relates to an innovative massage vibrator 900, specifically designed for medical applications involving internal devices within a patient's stomach. The massage vibrator 900 features an advanced Poron shock absorber 902, which serves to mitigate the transmission of excessive vibrational forces to sensitive internal tissues, thereby ensuring patient safety and comfort during the procedure.

Encased within a robust plastic casting 904, the massage vibrator 900 is engineered to be both durable and biocompatible, suitable for medical use. The plastic casting 904 houses a powerful magnet 906, which is a critical component of the device's functionality. The magnet 906 is used to create a controlled magnetic field capable of interacting with a corresponding magnetically responsive device located within the stomach of the patient. This interaction allows for precise manipulation and positioning of the internal device, facilitating targeted therapeutic interventions without the need for invasive surgical procedures.

The shaft 908, attached to the massage vibrator 900, is specifically designed to transmit controlled vibrations through the walls of the stomach to the internal device. These vibrations can be adjusted in intensity and pattern to perform various therapeutic functions, such as breaking down stomach contents or stimulating internal tissues to enhance digestive processes or relieve discomfort.

Additionally, the design of the massage vibrator 900 includes considerations for the unique environment of the stomach. The materials used for the shaft 908 and the external surfaces of the vibrator are selected for their resistance to stomach acids and their ability to operate effectively in the variable temperature and pH conditions found within the human digestive system.

This novel application of vibrational therapy, facilitated by magnetic interaction between the massage vibrator 900 and an internal device, opens up new possibilities in the treatment and management of gastrointestinal disorders, offering a non-invasive alternative to traditional treatment methodologies. This could revolutionize how certain stomach ailments are treated, providing relief and recovery options that were previously unattainable with conventional medical technologies.

Overall, the vibrating implant and its associated external vibrating magnet present a versatile and innovative solution for reducing satiety in a patient. The design considerations ensure safety, ease of use, and adaptability to different scenarios, making it a valuable addition to modern obesity treatments.

FIG. 8 illustrates an external vibrating magnet, a key component of the disclosed embodiment, designed to synergize with an internal device located within a patient's body. This external vibrating magnet can be configured as a massage vibrator, offering a versatile and non-invasive therapeutic tool.

In one embodiment, the external vibrating magnet is configured to allow the manipulation and control of an internal device within the stomach or other regions of the body. The massage vibrator function of the external magnet facilitates precise vibrational therapy that can be directed to specific internal areas, promoting enhanced therapeutic outcomes. This is particularly beneficial in medical treatments requiring the stimulation of tissues or aiding in the breakdown of contents within the gastrointestinal tract.

The design of this external vibrating magnet involves a control module configured to ensure that the vibrations produced are of the appropriate amplitude and frequency to effectively interact with the internal device without causing discomfort or harm to the patient. It employs a high-grade magnet that produces a controlled magnetic field strong enough to penetrate the body's natural barriers and reach the internal device. This magnetic interaction allows for the precise alignment and movement of the internal device, guided by the external inputs from the massage vibrator.

Moreover, the external vibrating magnet utilizes materials that are durable yet safe for use in close proximity to human skin and internal organs. Its ergonomic design allows for easy handling and application by patients and/or medical professionals, ensuring that the device can be used in various clinical settings and scenarios with ease.

In another embodiment, the external vibration device designed to function autonomously, without the need to physically move an intragastric magnetic implant. This device can operate in a variety of modes, either independently or in coordination with the implant, enhancing the therapeutic options available for weight management and digestive health.

Firstly, according to an embodiment it can be used for a period, such as two months, prior to the placement of the intragastric implant and can continue to be used after the implant has been removed. This method allows for a seamless transition in therapeutic modalities, providing consistent stimulus to the gastric region, which can be important in managing patient conditions effectively over time.

In a further embodiment, the device can be used in conjunction with the implant in a scheduled manner throughout the day. For example, the device might be used independently for breakfast, combined with magnetic distension at lunch, and then both methods might be employed together at dinner. This flexibility allows for tailored therapeutic approaches that can adapt to the patient's daily routine and nutritional intake.

Moreover, the vibration device can target specific areas such as the intestines and stomach, either separately or simultaneously with the implant, to provide targeted distension. This dual-functionality promotes a more comprehensive approach to managing gastrointestinal functions and enhancing the overall efficacy of the treatment.

To integrate modern technology, the vibration device can also be controlled through an application supported by artificial intelligence and remote monitoring systems. This integration not only facilitates user-friendly operation but also enables real-time adjustments and monitoring, potentially improving patient outcomes through personalized data analytics.

In another aspect of this embodiment, the external vibration device could incorporate an external magnet as part of the vibrating mechanism. Furthermore, the design of the external vibration device may utilize various sizes. For instance, a smaller device capable of higher frequency vibrations could be advantageous for specific therapeutic needs. Additionally, the concept of an external air pressure balloon could be explored as an alternative or adjunct to the vibration device, broadening the scope and applicability of the device within medical treatment frameworks.

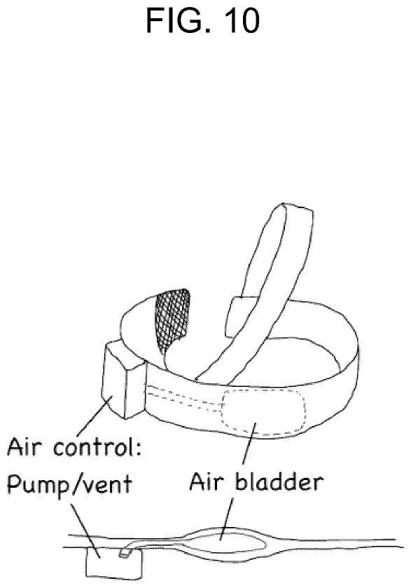

FIG. 10 discloses an external device containing an air bladder. The air bladder functions as an airtight pouch connected to an air-controlled unit responsible for regulating the inflation and deflation of the pouch. Housed within a box, the air-control unit is integrated with both the box and the pouch onto a belt that encircles the stomach. The vertical positioning of the belt is secured by a shoulder strap.

The belt's vertical position is fixed by the shoulder strap. The air-control unit include the air-pumping mechanism and release valve(s) controlled by on-board electronics that is also remotely connected to smart devices for customizable control and stimulations.

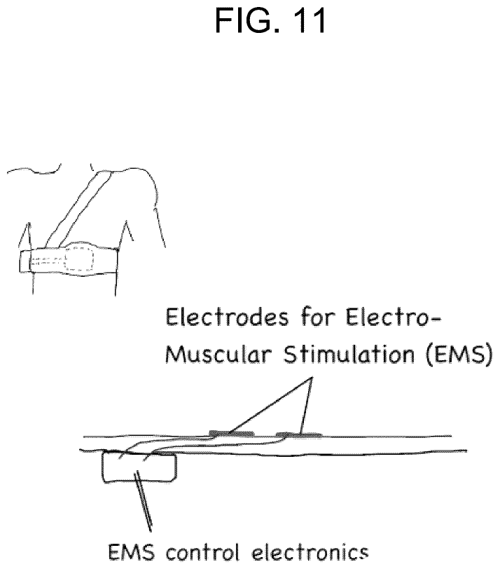

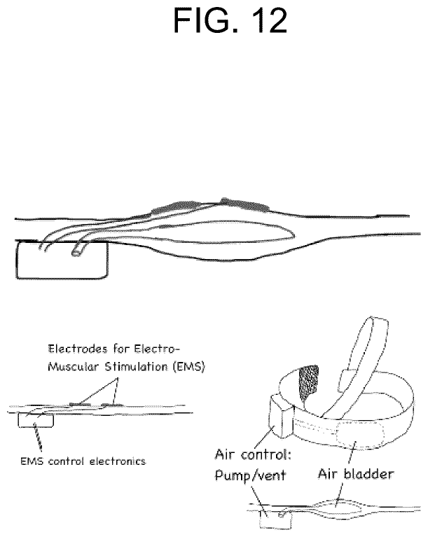

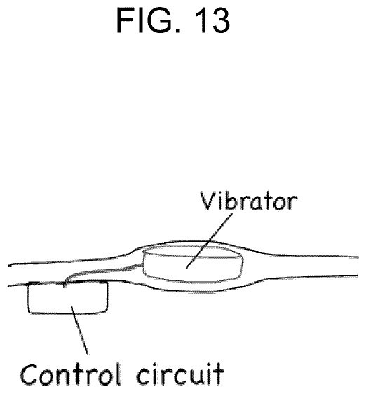

FIG. 11 discloses EMS (Electro-Muscular Stimulation) electrodes for stimulation, along with EMS control electronics integrated into a belt. EMS has the potential to stimulate gastric neural responses. Within the belt, two electrodes crafted from corrosion-resistant and biocompatible metals are positioned on the inner surface. These electrodes are linked to EMS control electronics, which are housed within a box integrated into the belt. The control electronics can be remotely accessed and managed by smart devices to control and regulate the stimulations. FIG. 12 discloses a hybrid approach, where both the air bladder and EMS can be integrated together on the belt. FIG. 13 discloses a vibrator embodiment where a disk-shaped mechanical vibrator is housed in the belt and controlled by the control electronics housed in a box integrated to the belt. The vibrator is fixed to the belt to minimize its lateral movement. Its surface vibrates back and forth to deliver gentle punches to the stomach.

In a further embodiment Both the air bladder and EMS can be integrated together on the belt. A disk-shaped mechanical vibrator is housed in the belt and controlled by the control electronics housed in a box integrated to the belt. The vibrator is fixed to the belt to minimize its lateral movement. Its surface vibrates back and forth to deliver gentle punches to the stomach.

The use of external vibrational devices can be enhanced by integrating them with a mobile app, allowing for precise control and customization. Additionally, combining these devices with virtual reality experiences can significantly amplify their effectiveness by providing an immersive environment that enhances the sensory experience.

By incorporating virtual reality, users can engage in tailored scenarios that complement the vibrations, creating a holistic approach to therapy or relaxation. This integration not only personalizes the experience but also maximizes the potential benefits by synchronizing visual, auditory, and tactile stimuli. As a result, users can achieve deeper states of relaxation, improved focus, and enhanced overall well-being.

Within the air-control unit, there is an air-pumping mechanism and release valve(s) controlled by onboard electronics. These electronics are also remotely connected to smart devices, allowing for customizable control and stimulation.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A vibration device, comprising:
a shock absorber with a plastic casing; and
a shaft containing a vibrating transmitter which sends controlled vibrations through the walls of the stomach to an internal device, wherein the internal device further comprises a balloon with a proximate end and a distal end; and
a magnet on the proximate end configured to expand the deflated structure to its inflated shape by magnetic attraction to the one or more magnets at the distal end.

2. The vibration device of claim 1, further comprising:
a stent within the balloon;
a core inner tube going through a middle of the balloon from the proximate end to the distal end;
one or more magnets at the distal end; and
a core outer tube outside the core inner tube.

3. The vibration device of claim 2, further comprising a vest that is connected to the shaft.

4. The vibration device of claim 3, further comprising a control module configured to ensure that the vibrations produced are of an amplitude and frequency determined by the control module.

5. A method, comprising:
providing a shaft containing a vibrating transmitter;
sending, via the vibrating transmitter, controlled vibrations through the walls of the stomach to an internal device, wherein the internal device further comprises a balloon with a proximate end and a distal end; and
expanding a deflated structure of the balloon device to an inflated shape by magnetic attraction to one or more magnets at the distal end.

6. The method of claim 5, wherein the vibrating transmitter further comprises a magnet than interacts magnetically with the internal device.
